(19) United States
(12) Patent Application Publication
(10) Pub. No.: US 2008/0188896 A1
(43) Pub. Date: Aug. 7, 2008

(54) Anchoring System for Fixing Objects to Bones

(75) Inventor: Lionel C. Sevrain, Plymouth, MA (US)

Correspondence Address: BURNS & LEVINSON, LLP, 125 SUMMER STREET, BOSTON, MA, 02110, US

(73) Assignee: OrthoPlex, LLC, Lincoln, MA (US)

(21) Appl. No.: 11/837,937

(22) Filed: Aug. 13, 2007

Related U.S. Application Data
(63) Continuation-in-part of application No. 10/275,710, filed on Nov. 8, 2002, now abandoned.
Application No. 10/275,710 is a continuation-in-part of application No. 10/358,398, filed on Feb. 5, 2003.
Application No. 10/358,398 is a continuation-in-part of application No. 10/296,392, filed on Sep. 15, 2003, now Pat. No. 7,008,427.

(51) Int. Cl. A61B 17/58; A61B 17/56

(52) U.S. Cl. 606/280; 606/301

(57) ABSTRACT

A spinal plate system that allows for increased spinal stability is provided. In an embodiment, a spinal plate system is provided that includes first and second slidable plates coupled together. Each of the first and second slidable plates includes first and second fasteners that are obliquely oriented relative to the corresponding slidable plate. The first and second fasteners are fixed to each other at their distal ends thereby forming a triangular frame consisting of the corresponding slidable plate and the first and second fasteners. In addition, a fastener guide is provided that facilitates the accurate placement of the first and second fasteners for each of the first and second slidable plates.

### CROSS REFERENCE TO RELATED APPLICATIONS

This application is a continuation-in-part of US Non-Provisional Patent application, Ser. No. 10/275,710 filed May 25, 2001, which is incorporated herein by reference, that was the US National Phase of international application PCT/CA01/00751 filed May 25, 2001 which designated the US and which claims priority to Provisional Application, Ser. No. 60/206,811 filed on May 25, 2000. and is also continuation-in-part of US Non-Provisional Patent application, Ser. No. 10/358,398, which is incorporated herein by reference, filed Feb. 5, 2003 that is a continuation in part of U.S. Ser. No. 10/296,392 filed Nov. 25, 2002 which is a National Entry of PCT/CA01/00739 filed on May 25, 2001 and claiming priority to US Provisional Applications Ser. No. 60/206,810 filed on May 25, 2000 and Ser. No. 60/264,309 filed Jan. 29. 2001

### BACKGROUND OF THE INVENTION

1. Field of the Invention

The present invention relates generally relates to a bone fixation systems. In particular, the present invention relates to a plate for a spinal fixation system for attaching various objects, such as prostheses or implants to bones, including for anchoring spinal instrumentations to vertebrae of the human rachis and may be used in conjunction with a drilling and aiming guide.

2. Description of the Related Art

U.S. Pat. No. 5,366,455 issued to Dove et al. on Nov. 22, 1994, U.S. Pat. No. 5,672,175 issued to Martin on Sep. 30, 1997, U.S. Pat. No. 5,733,284 issued to Martin on Mar. 31, 1998 and U.S. Pat. No. 5,437,672 issued to Alleyne on Aug. 1, 1995 disclose devices for anchoring various supports, e.g. spinal orthoses, to the rachis, these devices being adapted to obviously extend outwardly of I the spinous process or canal and thus of the spinal cord.

U.S. Pat. No. 5,800,433 issued to Benzel et al. on Sep. 1, 1998 and U.S. Pat. No. 5,954,722 issued to Bono on Sep. 21, 1999 teach anchoring systems having screws which are angled such as to converge towards each other.

U.S. Pat. No. 5,904,683 issued to Pohndorf et al., on May 18, 1999 and U.S. Pat. No. 5,980,523 issued to Jackson on Nov. 9, 1999 disclose anterior cervical vertebrae stabilizing devices held in place by various types of screws.

To try prevnting the screws from loosening, various systems have been used, such as directing the screws along different orientations (e.g. diverging or converging); providing a locking mechanism on the screw (e.g. counter-nut); modifying the screw's thread (height and depth); engaging each screw to two tissues having different densities; etc.

A vertebra disc may be subject to degeneration caused by trauma, disease, and/or aging. The degenerated vertebrae disk may have to be partially or fully removed from a spinal column. Partial or full removal of a vertebrae disk may destabilize the spinal column and may alter the natural separation between adjacent vertebrae. Individual nervew eminate at the brain and pass down the nmerve sheath located poseterior to the spine. These individual nervew eventually pass through thte intervertebral space to various body member, i.e., hands, feet, etc. Maintaining a natural separation between vertebrae is essential in order to prevent pressure from being applied to nerves that pass between the adjacent vertebrae. Excessive pressure applied to the nerves may cause pain and/or nerve damage. During a spinal fixation procedure, a spinal implant may be inserted in order to restore space created by the removal or partial collapse of a vertebral disk. A spinal implant may maintain the height of the spine, restore stability to the spine, and prevent nerve damage. Bone growth may fuse the implant to adjacent vertebrae thereby facilitating long term pain relief.

A spinal implant may be inserted during a spinal fixation procedure using an anterior, lateral, or posterior spinal approach. In some situations especially in the anterior cervical approach may result in less difficult surgery resulting in less muscle damage, less tissue damage, and/or less bone removal than other approaches.

A discectomy is a neurosurgical procedure to remove, or partially remove, a defective and/or damaged vertebrae disk. A discectomy creates a disk space so that a spinal implant may be inserted into the disk space created between one or more pair of vertebrae.

One or more spinal plates may be coupled to vertebrae after insertion of one or more spinal implants. A spinal plate may help stabilize the vertebrae and inhibit back out of the spinal implant from between the adjacent vertebrae. The spinal plate may share the compressive load applied to one or more spinal implants inserted between the vertebrae. Fasteners, e.g., bone screws may be used to couple the spinal plate to the vertebrae itself Spinal plates may be used to stabilize sections of cervical spine and/or sections of lumbar spine.

The current art for fastening plates to spinal vertebrae has at least one major problem: loosening of the fasteners. Loosening of the fasteners that may lead to compression and/or perforation of adjacent tissue. This loosening may be caused by resultant biomechanical forces, micro motions of the plate system and fasteners, or the poor quality of osteoporotic bone. The inability for a bone screw to properly purchase into the osteoporotic bone is a major problem for older patients, leading to loosening of the spinal plates and increasing the possibility of having the older patient undergo a second operation to reposition or otherwise retighten the spinal plates.

Therefore what is needed is a spinal plate that is securely affixed to the bone regardless of the quality of the bone, prevents the back out of the fasteners and/or the loss of purchase of a fastener due to the poor quality of the bone itself.

### SUMMARY OF THE INVENTION

A spinal plate system that allows for increased spinal stability is provided. In an embodiment, a spinal plate system is provided that includes first and second slidable plates coupled together. Each of the first and second slidable plates includes first and second fasteners that are obliquely oriented relative to the corresponding slidable plate. The first and second fasteners are fixed to each other at their distal ends thereby forming a triangular frame consisting of the corresponding slidable plate and the first and second fasteners. In addition, a fastener guide is provided that facilitates the accurate placement of the first and second fasteners for each of the first and second slidable plates.

### BRIEF DESCRIPTION OF THE DRAWINGS

Aspects of the present invention are pointed out with particularity in the appended claims. The present invention is illustrated by way of example in the following drawings in which like references indicate similar elements. The following drawings disclose various embodiments of the present invention for purposes of illustration only and are not intended to limit the scope of the invention. For purposes of clarity, not every component may be labeled in every figure. In the figures:

FIG. 1 is a schematic cross-sectional plan view of a bridging plate mounted to a lumbar vertebra using an anchoring system in accordance with the present invention;

FIG. 2 is a schematic anterior perspective view of a bridging plate mounted to a pair of cervical vertebra using' the anchoring system in accordance with the present invention;

FIG. 3 is a front view of the anchor system described herein;

FIG. 4 is a plan view of the anchor system depicted in FIG. 3;

FIG. 5 is a side view of the anchor system depicted in FIG. 3;

FIG. 5A is a perspective view of the anchor system depicted in FIG. 3;

FIG. 6 is a top view of a fastener guide compatible with the anchor system depicted in FIG. 3; and

FIG. 7 is a depiction of one key structure compatible with the anchor system described herein.

### DETAILED DESCRIPTION

The following detailed description sets forth numerous specific details to provide a thorough understanding of the invention. However, those skilled in the art will appreciate that the invention may be practiced without these specific details. In other instances, well-known methods, procedures, components, protocols, processes, and circuits have not been describe in detail so as not to obscure the invention.

A spinal plate system that may be used to stabilize a portion of a spinal column and a method of use are described in more detail below. The spinal plate system includes first and second plates that are slidably coupled to one another and allow for motion of each of the plates relative to the other. Each of the first and second plates is secured to the bone by an obliquely oriented post and screw system wherein the post and screw are oriented so as to converge to one another. The post includes an oblique threaded hole that receives the distal portion of the screw. In this manner, the post and screw interlock with one another such that the plate, the screw and the post form a structurally stable triangular frame. This triangular frame is inherently structurally stable and strong and therefore does not rely on the bone quality for its inherent stability or strength. Advantageously, this triangular frame structure prevents screw loosening and back out, increases the pullout strength of the plate, allows for removal of the plate due to the threaded connections, and shortens patient immobilization since the strength of fixation is now independent of bone quality.

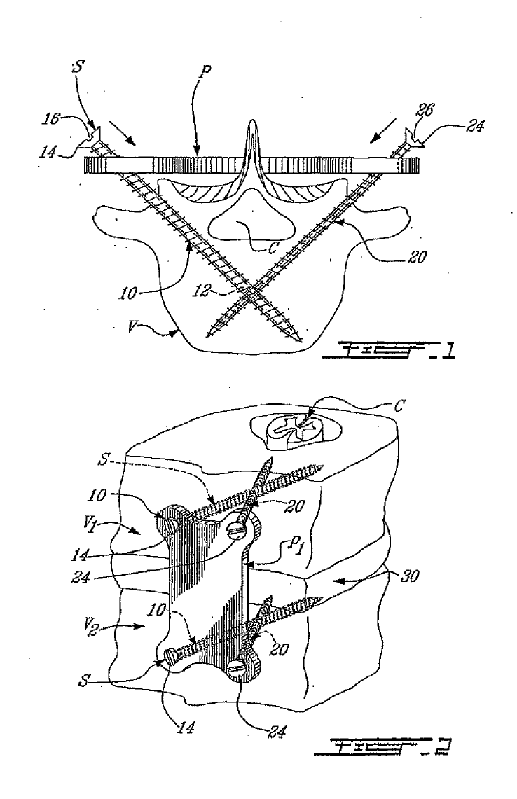

FIG. 1 illustrates an anchoring system in accordance with the present invention which is herein schematically shown in an engaged position. to a lumbar vertebra V of the human rachis for holding firmly thereagainst a spinal prosthesis or spinal instrumentation, such as a support plate P, from a posterior approach.

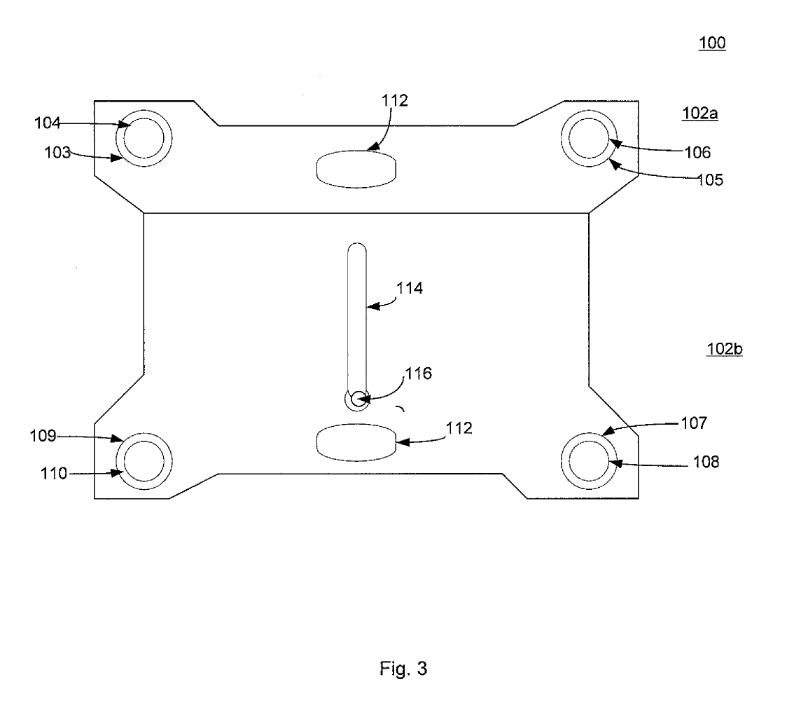

FIG. 2 illustrates two anchoring systems S that hold an object( such as a cervical plate pi that has been positioned after classical anterior or antero-lateral approach of the cervical spine and that is herein used to 1 ink together two or more adjacent vertebrae, such as vertebrae V1 and V2′ For instance, when a cervical disk is anteriorly removed. (see 30 in FIG. 2) from between two adjacent vertebrae, it is known to fuse both these vertebrae together to provide stability to the rachis. This can be done by securing plates to the vertebrae. To replace the disks, a spinal prosthesis may be installed and such a prosthesis typically comprises a pair o˜ upper and lower plates secured with screws respectively to ,the upper and lower vertebrae between which the disk has been -removed with -a prosthetic disk being provided between these vertebrae and which is held in place by the plates. U.S. Pat. No. 5,258,031 issued to Salib et al. on Nov. 2, 1993 and U.S. Pat. No. 6,001,130 issued to Bryan et al. on Dec. 14, 1999 disclose known examples of intervertebral disk prostheses which are secured to adjacent vertebrae using screws which extend therein. In these cases, one screw is used to mount each of the upper and lower s˜pport assemblies to its respective vertebra. On the other hand, U.S. Pat. No. 5,755,796 issued to Ibo et al. on May 26, 1998 teaches a cervical disk prosthesis wherein each of the upper and lower plates are secured to its respective vertebra by way of a pair of screws which are horizontally spaced apart from each other. The prosthesis is secured anteriorly of the rachis and the screws thereof are short enough not to reach the spinal cord.

In other cases however (posterior approach), the screws are longer and are generally directed on each side of the spinous canal in order to obviously prevent damage to the spinal cord.

Therefore, the present anchoring system S (for each of FIGS. 1 and 2) comprises first and second screws 10 and 12, respectively, which are adapted to be introduced in the vertebra at an angle and convergingly towards each other, as seen in FIG. 1. In the illustrated embodiment, the first screw 10 is larger than the second screw 20 and defines near its distal end an oblique threaded through opening 12. The two screws 10 ind 20 have flat head 14 and 24 which define a depression which is shaped to be engaged by a screwdriver, or the like, for inducing torque thereto and causing the screws 10 and 20 to rotatably engage the vertebra V and gradually advance translationally thereinto, in a conventional manner. In FIG. 1, these depressions are slots 16 and 26 for use with a flat blade screwdriver, although the depressions could be cruciform, square, hexagonal, torx, etc., shaped.

The second screw 20 has a threaded stem of which at least a distal section is smaller the first screw 10 as the second screw 20 is adapted to extend through the opening 12 of the first screw 10 such as to threadably engage the same. Indeed the male threads of the second screw 20 are designed female threads of the opening 12 of the first screw 10 thereby securing together the distal ends of the two screws 10 and 20. With these distal ends so engaged and with the screws 10 and 20 extending in a convergent attitude, there is resistance, where the two screws 10 and 20 are engaged together, to the forces which tend to cause the screws to gradually loosen, whereby it is impossible for the screws 10 and 209 to loosen (unless the vertebra V itself is destructed where it is engaged by the screws 10 and 20, or unless one of the screws 10 and 20 breaks).

In fact, the first screw 10 acts as a nut for the second screw 20, and this within the vertebra V itself in FIGS. 1 and 2 (as opposed to conventional nuts which normally engage the screw or bolt on the outside of the object through which the screw or bolt extends.

The first screw 10 is preferably provided at its head 14 with indicia (color, index, etc.) to indicate the position of its distal end so that the position or orientation of its threaded opening 12 can be more easily determined thereby facilitating the introduction of the second screw 20 therethrough. An aiming system maybe used as a guide during the screwing process. For instance, to ensure an accurate aiming of the two screws 10 and 20 and their relative engagement, a template may be used to guide both screws from an initial predetermined spacing, along a given plane and along predetermined angles. Alternatively, a neuro-navigation apparatus can also be used, that is a computer software capable of transposing digitized data taken from a pre-surgery medical imagery of, the stereotactic space in which the surgeon will operate.

The obliqueness of the threaded opening 12 through the first screw 10 depends on the angle, that is on the spacing on the plate P/P′ between the two screws 10 and 20 (i.e. generally the spacing between their heads 14 and 24) in a horizontal plane, as well as the directions of the screws 10 and 20 in the sagittal plane.

The two screws 10 and 20 extend in holes defined in the plate P/P′, and would normally have their heads 14 and 24 in abutment with the proximal surface of the plate P/P′ (as in FIG. 2, but as opposed to the schematic illustration of FIG. 1 where the heads 14 and 24 are shown spaced from the plate P but simply for illustration purposes). The holes in the plate P/P′ are typically angled so as to ensure the crossing of the screws 10 and 20 at a precise location in the vertebra V and so permit the threaded engagement of the second screw 20 in the opening 12 of the first screw 10 once the first screw 10 is completely fixed (i.e. screwed in the vertebra V) and once the position of its opening 12 is determined by way of the indicia on its head 14.

The two screws 10 and 20 and the plate P/P define a triangular frame (which is well shown in FIG. 1) which is rigid, closed and locked in place, having its, components locked together in a solid medium, i.e. the vertebra V, whereby expulsion of the screws 10 and 20 from the vertebra V is opposed. Each of the three components 10, 20 and P/P′ of this frame is integral to the preceding component and to the next component. The triangulation screwing process is a concept based on the principle that a frame is much stronger than an, open structure. By connecting two screws at their distal ends, it becomes possible to create such a frame. This triangular configuration is also, convenient as it allows the anchoring system S, in addition to firmly securing the plate pip to the vertebrae V, V1 and V2 and preventing a loosening of the plate P/P′ with respect to these vertebrae, to extend around the spinal process or canal C and thus around the spinal cord when the plate P/P′ is, for instance, installed posteriorly (see FIG. 1) .

In the case of the use of the anchoring system S to install the plate P′ onto the adjacent vertebrae VI and V2 of the cervical rachis (FIG. 2), along an anterolateral path, for instance following the removal of an herniated disc, osteosynthesis can be realized by fixing a plate P′ (e.g. a “Senegas”-type plate) with anchoring two systems S, as in FIG. 2. The plate P′ is. centered about the intersomatic space 30, which is devoid of its natural disc, the latter having been replaced by a disc prosthesis. The first screws 10 are then positioned in the left holes 14 of the plate P′, along an anteroposterior axis or slightly obliquely from the left to the right, as the screwdriver will be hindered by the thickness of the oseo-tracheal axis (displaced to the left), before the first screws 10 are screwed through the left holes 14 and into the vertebrae V1 and V2. Once the first screws are completely set inot the vertebrae V1 and V2, and properly positioned using their indicia, the two second screws 20 can be screwed through the right holes 14 of the plate P′ and into the vertebrae V1 and V32, which is easier than for the first screws 10 as the second screws 20 can be more easily inclined from right to left as the jugulo-carotid bundle is not as obstructive. A scopic control can ensure the proper engagement of the two screws 10 and 20 of each anchoring system S.

In the case of the plate P of FIG. 1 secured posteriorly at least to the vertebra V with the anchoring system S, the determination of the entry points in each of the pedicles of the, vertebrae V can be realized according to Roy-Camille. The plate P, or a linking rod, is then positioned horizontally and transversely such that its holes are opposite the pre-determined entry points. The screws 10 and 20 are then installed as above to form with the aforementioned triangular frame. This triangular frame, which is rigid and intra-vertebral, can then be solidified to upper and lower frames using plates or rods, in a conventional manner.

In order to facilitate the engagement of the second screw 20 into the first screw 10, the opening 12 in the first screw 10 may instead of being threaded, have the form of a spherical socket that rotatably accommodates a ball. A hole extends, typically diametrically, through this ball and defines an interior thread, that is a female thread that can be screwably engaged by the male thread of the second screw 20. Therefore, the ball could rotate within the socket to allow for a correction in the direction of the second screw 20 relative to the first screw 10; in other words, if the second screw 20 is slightly off target in its orientation with respect to the hole defined in the ball of the first screw 10, the ball may be slightly rotated to align the longitudinal axis of its hole with the axis of the second screw 20.

It is also contemplated to provide a threadless opening in the first screw 10 instead of the threaded opening 12; in such a case, the opening would be self-tapping in that the male threads of the second screw 20 would tap a thread in the opening of the first screw 10 upon rotary engagement therein. Similarly, the above-mentioned ball could also be threadless and self-tapping. Furthermore, the first screw 10 could be replaced by a threadless pin or nail that would be translationally insertable in the bone and that would define an opening (threaded or self-tapping) at its distal end for receiving the second screw 20.

It is further contemplated to use elongated anchoring members other than the above-described and herein illustrated screws 10 and 20, as well as other means of securing the distal ends of such anchoring members together. For instance, the screws 10 and 20 could be replaced by threadless pins or nails that would be translationally inserted in the bone. In such a case, the distal end of a first one of the anchoring members could define an opening, such as an elongated slot, through which the distal end (which would, for instance, be flat) of a second one of anchoring members could be inserted. A locking mechanism between the two distal ends could take the form of a lateral pin extending from the distal end of the second anchoring member which, after having been passed beyond the elongated slot in the first anchoring member, would be rotated ¾ turn such as to extend behind the body of the first anchoring member thereby locking the distal ends together. Such a pin could be embodied in the distal end of the second anchoring member being L-shaped or T-shaped or defining a barb-shaped extension.

The common feature is two elongated members insertable in the bone and having distal ends capable of being interlocked for preventing unwanted withdrawal of any of the two elongated members from the bone.

Although the present anchoring system S has been shown herein in use to secure a plate P/P′ to one or more lumbar (FIG. 1) or cervical (FIG. 2) vertebrae, the system S can also be used to plates, for instance to secure rods instead of plates, for instance to the dorso-lumbar rachis, and in fact can be used to affix various objects to various bones of the body, and not only to the rachis. The system S can thus be used not only as described above and herein illustrated but also in orthopaedic, in neuro-surgical, otorhinolaryngological, maxilla-facial and stomatological applications.

Every component of the anchoring system S is made of biocompatible material or of a material capable of being so coated.

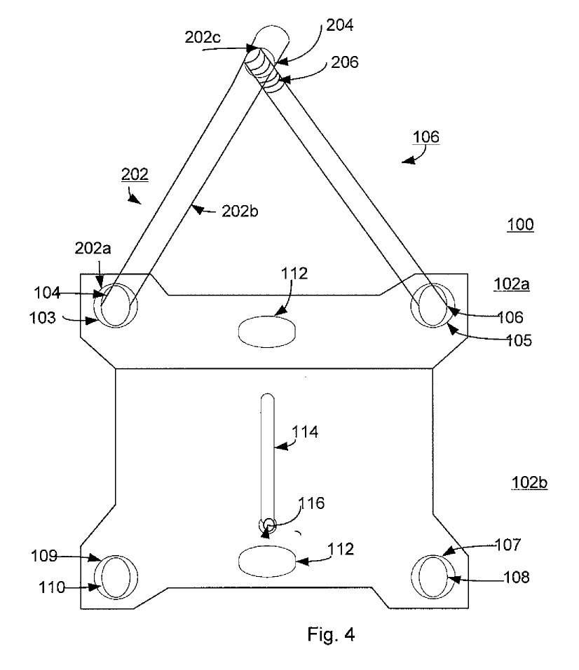

According to another embodiment of the present invention depicted in FIG. 3, a sliding plate system 100 includes a first plate 102a and a second plate 102b. Plate 102a includes a pair of holes 103 and 105 that are configured to receive a post 104 and a screw 106 respectively. In one embodiment, the hole 103 includes a key feature, such as a flat side forming a D shaped opening. Post 104 also includes this key feature and is thereby oriented in a desired manner when inserted into the opening 103. Plate 102a may also include an attachment opening 112 that is sized and configured to receive an attachment element from a fastener guide, which is described in more detail below. Plate 102a may also include a pin 116 extending therefrom. The pin 116 may also include a threaded portion that extends below the plate 102a to which a spinal prostheses, allograph, or autograph may be attached to and used to restore a desired intervertebral height between a pair of adjacent vertebrae. The attachment 106 may also be a screw which holds an elongated member used to restore the space between adjacent vertebrae. The attachment 106 may be a screw which holds an allograph member used to restore the space between adjacent vertebrae.

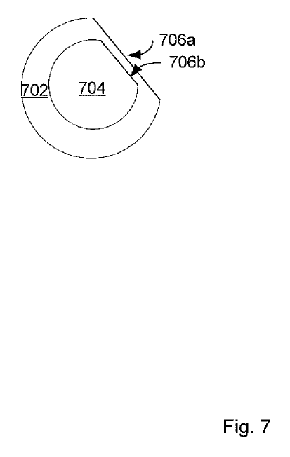

Plate 102 b includes a pair of holes 107 and 109 that are configured to receive a post 108 and a screw 110, respectively. In one embodiment, the hole 107 includes a key feature, such as a flat side forming a D shaped opening as depicted in FIG. 7, where the key feature includes a rounded portion of the hole 702 and post 704 and flat portion 706a and 706b. Post 108 also includes this key feature and is thereby oriented in a desired manner when inserted into the opening 107. Plate 102b may also include an attachment opening 112 that is sized and configured to receive an attachment element from a fastener guide, which is described in more detail below. Plate 102b also includes an elongated opening 114 that is configured to receive the pin 116. As discussed above, the plates 102a and 102b may slide relative to one another and pin 116 in conjunction with elongated opening 114 are operative to limit the motion of the two plates relative to one another.

In general, the holes 103, 105 are oriented at an oblique angle and in general are converging toward one another. In particular, the holes are oriented such that the center line extending from hole 103 will intersect the center line from opening 105. Similarly, plate 102b includes holes 107 and 109 are oriented in a similar fashion such that center line extending from hole 107 will intersect with the center line from hole 109. In this way, the post 104 and screw 106 will intersect with one another as will post 108 and screw 110.

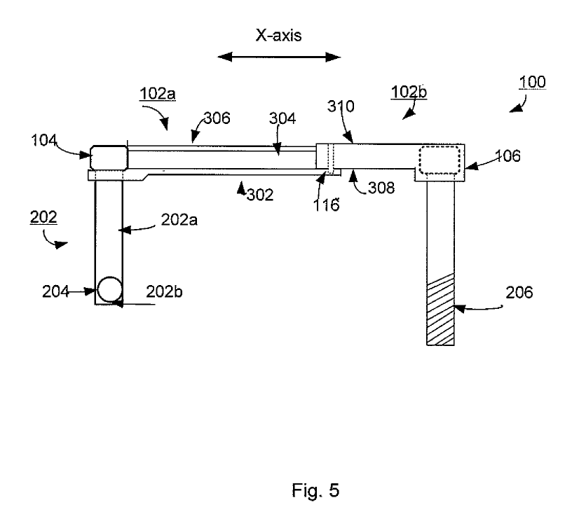

As depicted in FIG. 4, Post 104 has a head portion 202a, a shaft portion 202b, and a distal portion 202c. An oblique hole 204 is formed through the distal portion of the post 104. The oblique hole 204 includes internal threading that is complementary to the threads of threaded portion 206 of screw 106. The oblique hole 204 is oriented such that the corresponding screw is received and is properly seated such that the complementary threads can engage one another and the screw can be threaded into the oblique hole. In this manner, the post 104 and screw 106 interlock with one another and it is not necessary for the screw to purchase into the bone itself The plate 102a, post 104 and screw 106 thus form a triangular frame that is both structurally stable and strong. Plate 102b includes similar structures with regard to holes 107 and 109, post 108 and screw 110. Thus, plate 102b also forms a triangular frame that is also both structurally stable and strong. The triangular frame that is depicted is an equilateral triangle. However, the triangle may be a right isosceles triangle, a regular isosceles triangle, or a scalene triangle. The actual form of the triangular frame is dependent on the width of the bone and the proximity of other body structures such as arteries, nerves, the esophagus, or other internal organs.

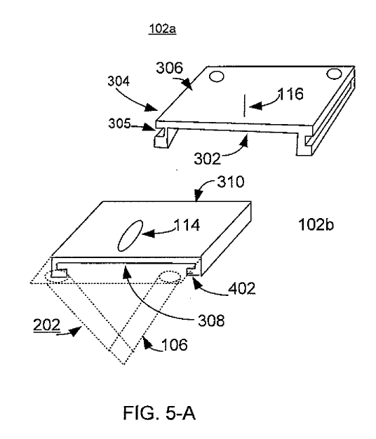

As discussed above, the plates 102a and 102b are able to slide relative to one another along the x-axis depicted in FIG. 5. As farther depicted in FIGS. 5 and 5a, plate 102a includes bottom surface 302, top surface 306, and side surface 304 wherein side surface 304 includes indentation 305. Plate 102b includes bottom surface 308, top surface 310, and a side surface 312 that includes a tab portion 402 sized and configured to extend into, and be slidably received by, indentation 305 of side surface 304 of plate 102a. In particular, the bottom surface 308 is disposed above bottom surface 302 and the top surface 310 is disposed above the top surface 306. Pin 116 extends above the top surface 306 where it is received within the elongated hole 114 and thereby limits the motion of plates 102a-b with respect to one another. As discussed above, in one embodiment, the pin 116 may extend below bottom surface 302 and use a threaded portion or other attachment means to secure a spinal prostheses, autograph, or allograph to the bottom surface 302 when used to maintain the intended spacing or to restore a desired intervertebral height between a pair of adjacent vertebrae.

In general, the sliding plates 102a-b, the posts 104 and 108, and screws 106 and 110 are constructed from 6AL4V Titanium ELI grade 5 titanium per ASTM-F-136-92. The sliding plates 102a-b may also have an curved shape conforming to the vertebrae shape both in the longitudinal axis (i.e., lordotic curvature) and the transverse axis. The curvature of the curve shape may be such that the sliding plates 102a-b are able to be securely fastened to vertebrae having both a radial curvature as well as a lordotic curvature.

As discussed above, both the holes 103 and 107 and the posts 104 and 108 include a key structure such as a flat portion forming a “D” shaped opening. This allows for a particular orientation of the post to be achieved in a consistent manner. Because it is important that the oblique opening 204 be oriented to receive the threaded portion 206 of the corresponding screw a consistent method of achieving the proper orientation is necessary. By forming the post with the appropriate key structure, the proper consistent orientation may be achieved. Any key structure may be used, and the “D” shaped hole used herein is but one example. Without limitation, any suitable key structure known in the art may be used.

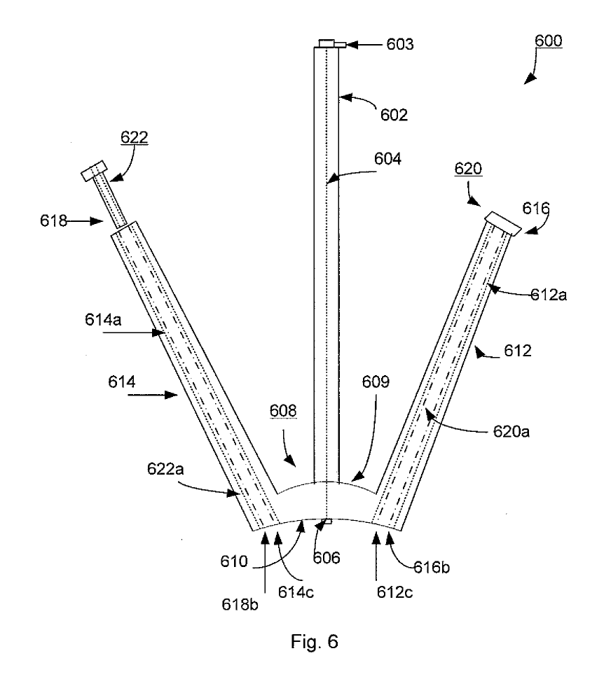

FIG. 6 depicts a fastener guide apparatus that is used with the spinal plate system described above to ensure that holes drilled into the spinal vertebrae are correctly oriented. In particular, the fastener guide 600 includes a base portion 608 that includes a top surface 609 and bottom surface 610. A central handle 602 extends outward from top surface 609. The central handle 602 includes a top portion 603 that is coupled via an interior shaft 604 to attachment element 606. As discussed above, the spinal plate system includes attachment openings 112 that are configured and sized to receive the attachment element 606 and thereby hold the spinal plate system securely to the fastener guide apparatus. Top portion 603 is coupled to attachment element 606 and in one embodiment is able to rotate attachment element 606 such that attachment element 606 may be inserted or removed from the attachment opening 112 in plates 102a-b.

The fastener guide 600 also includes first and second guide arms 612 and 614, respectively. The first and second guide arms 612 and 614 extend outward from the top surface 609 at an oblique angle, which is equal to the angle at which the post and screw are to be inserted into the bone. First and second guide arms each include a shaft 612a and 614a, respectively, that extends from the top surface 616 and 618 respectively, through the bottom surface 610. One shaft is provided with the key structure described above and in this embodiment, the attachment element 606 and the attachment opening 112 are configured and arranged such that the guide arm having the key structure will always be oriented over the opening 103 or 107 where the post will be disposed within that hole. In one embodiment, the shaft 612a and 614a will be accurate enough to allow an instrument to be inserted into the shaft and pass through the various openings in the spinal plate system described above. The instrument may be a drill, a fastener placement instrument, or a fastener tightener.

In another embodiment, the accuracy of shaft 612a and 614a is not sufficient and an additional insert is needed. In particular, guide insert 620 and 622 are used to provide the necessary increase in accuracy. Guide insert 620 is sized and dimensioned to fit securely within shaft 612 and has a shaft 620a that extends the length of the guide insert and communicates with the shaft opening 612c at the bottom surface 610. The shaft 620a is a highly accurately machined opening with high tolerances for the inner diameter. In general, shaft 620a is coaxially disposed within shaft 612a. Similarly, guide insert 622 is sized and dimensioned to fit securely within shaft 614 and has a shaft 622a that extends the length of the guide insert and communicates with the shaft opening 614c at the bottom surface 610. The shaft 622a is a highly accurately machined opening with high tolerances for the inner diameter and also includes any key structure present in the shaft 612a as discussed above. In general, shaft 622a is coaxially disposed within shaft 614a to allow an instrument to be inserted into the shaft and pass through the various openings in the spinal plate system described above. An instrument may be inserted into shaft 620a and 622a and may be a drill, a fastener placement instrument, or a fastener tightener as required. In another embodiment, the guide inserts 620 and 622 are used only for the drilling of the holes in the bone and the placement and tightening of the fasteners is performed using the shafts 612a and 614a.

It should be appreciated that other variations to and modifications of the above-described method and system for transferring and compressing medical image data may be made without departing from the inventive concepts described herein. Accordingly, the invention should not be viewed as limited except by the scope and spirit of the appended claims.

What is claimed is:

1. An anchoring system comprising:
An anchor member having a first and a second opening, said first opening having a predetermined structure;
a post having a complementary predetermined structure disposed through said first opening wherein said post is aligned in a particular orientation, said post being disposed at a first oblique angle with respect to the anchor member, said post having a head portion and an end portion, said end portion including a threaded hole extending obliquely there through, said threaded hole including first threads;
a screw having a head portion and a threaded end portion extending through said second opening, said screw being disposed at a second oblique angle with respect to the anchor member, wherein said threaded portion of said screw being complementary to said threads of said threaded hole, said threaded end portion extending into and received by said threaded hole, wherein said anchor member, post, and screw form a triangular frame;
said anchor member having third opening and a fourth opening, said third opening having a predetermined structure;
a second post having a complementary predetermined structure disposed through said third opening, said second post being disposed at a third oblique angle relative to said anchor member, said post having a head portion and an end portion, said end portion including a threaded hole extending obliquely there through;
a second screw having a head portion and a threaded end portion extending through said fourth opening, said screw being disposed at a fourth oblique angle relative to said anchor member, wherein said threaded portion of said screw being complementary to said third threads, said threaded end portion extending into and received by said threaded hole, wherein said anchor member, post, and screw form a triangular frame.

2. The anchoring system of claim 1 wherein said anchor member includes a first plate that includes said first and second opening and said first post and first screw and a second plate slidably coupled to said first plate, said second plate including said third and fourth openings and said second post and screw, wherein said first plate and said first post and said second screw form a first triangular form and said second plate and said second post and said second screw form a second triangular form.

3. The anchoring system of claim 2 wherein said first plate further including a top surface, a bottom surface, a pair of side surfaces each having indented portion, and a pin extending out from said top surface;
said second plate including a top surface, a bottom surface, and a side surface said side surface including tab portions sized and configured to slidably engage said indented portions of said first plate, wherein said bottom surface of said second plate is adjacent to the top surface of said first plate and wherein said first and second plates may slide relative to one another;
said second plate further including an elongated opening configured and arranged to receive said pin, wherein said elongated hole in conjunction with said pin limits the range of motion of said first and second plates relative to one another.

4. The anchoring system of claim 1, wherein said first and second oblique angles are all substantially equal and wherein said triangular frame is substantially an equilateral triangle.

5. The anchoring system of claim 1, wherein said first and second oblique angles are substantially equal and said threaded opening forms a right angle with said screw, and wherein said triangular frame is substantially a right isosceles triangle.

6. The anchoring system of claim 1, wherein said first and second oblique angles are substantially equal and said threaded opening forms an angle with said screw that is less than a right angle and wherein said triangular frame in an isosceles triangle.

7. The anchoring system of claim 1, wherein said first and second angles are substantially unequal, and wherein said triangular structure is a scalene triangle.

8. The anchoring system of claim 1, wherein said third and fourth oblique angles are all substantially equal and wherein said triangular frame is substantially an equilateral triangle.

9. The anchoring system of claim 1, wherein said third and fourth oblique angles are substantially equal and said threaded opening forms a right angle with said screw, and wherein said triangular frame is substantially a right isosceles triangle.

10. The anchoring system of claim 1, wherein said third and fourth oblique angles are substantially equal and said threaded opening forms an angle with said screw that is less than a right angle and wherein said triangular frame in an isosceles triangle.

11. The anchoring system of claim 1, wherein said third and fourth angles are unequal, and wherein said triangular structure is a scalene triangle.

12. The anchoring system of claim 1, further comprising said anchor member having a curved shapes, wherein the curved shape of said anchor member corresponds to an object to which the anchoring system is to be affixed.

13. The anchoring system of claim 2, further comprising said first and second plates having curved shapes, wherein the curved shape of each of said first and second plates corresponds to an object to which the anchoring system is to be affixed.

14. The anchoring system of claim 13, wherein said curved shape of said first or second plate corresponds to the lordotic curvature of one or more spinal vertebrae.

15. The anchoring system of claim 13, wherein said curve shape of said first or second plate corresponds to the radial curvature of one or more spinal vertebrae.

16. The anchoring system of claim 1, wherein said third and fourth angles are all equal and wherein said triangular frame is substantially an equilateral triangle.

17. The anchoring system of claim 1, wherein said third and fourth angles are substantially equal and said threaded opening forms a right angle with said screw, and wherein said triangular frame is substantially a right isosceles triangle.

18. The anchoring system of claim 1, wherein said third and fourth angles are substantially equal and said threaded opening forms an angle with said screw that is less than a right angle and wherein said triangular frame in an isosceles triangle.

19. The anchoring system of claim 1, wherein said third and fourth angles are unequal, and wherein said triangular structure is a scalene triangle.

20. The anchoring system of claim 1 further including said first plate having an attachment opening extending therethrough, said attachment hole configured and arranged to receive a guide attachment element.

21. The anchoring system of claim 1 further including said second plate having an attachment opening extending therethrough, said attachment hole configured and arranged to receive a guide attachment element.

22. The anchoring system of claim 1 wherein said first opening includes a predetermined structure and said corresponding post includes said predetermined structure wherein said post is oriented by said predetermined structure in a preferred orientation.

23. The anchoring system of claim 22 wherein said head portion of said post includes said predetermined structure.

24. The anchoring system of claim 1 wherein said third opening includes a predetermined structure and said corresponding post includes said predetermined structure wherein said post is oriented by said predetermined structure in a preferred orientation.

25. The anchoring system of claim 23 wherein said head portion of said post includes said predetermined structure.

26. The anchoring system of claim 1 wherein the pin includes a threaded portion capable of attaching to an allograph or a autograph or a spinal prosthesis intended to restore a desired intervertebral height between a pair of adjacent vertebrae.

27. A fastener guide for an object comprising:
a base portion including a bottom surface and a top surface;
a first guide arm extending from said top surface of said base portion said first guide arm having a top surface and having a shaft extending from said top surface of said first guide arm through said bottom surface of said base portion, said first guide arm having a first angle relative to said base portion, and said shaft having a key structure;
a second guide arm extending from said top surface of said base portion said second guide arm having a top surface and having a shaft extending from said top surface of said second guide arm through said bottom surface of said base portion, said second guide arm having a second angle relative to said base portion;
an arm disposed between said first and second guide arms and extending from said top surface of said base portion, said center arm having an attachment element disposed therethrough and extending beyond said bottom surface of said base portion, said attachment element being configured and arranged to releasably mate with an complementary attachment element of the object to be held by said fastener guide.

28. The fastener guide of claim 27 wherein said first guide arm shaft includes a key structure.

29. The fastener guide of claim 27, further comprising first and second guide inserts, said first and second guide inserts being sized and configured to be disposed coaxially within said first and second guide arm shafts, respectively, each of said first and second guide inserts having a central shaft disposed therethrough, and said shafts in said first and second guide inserts being in communication with said shafts in said first and second guide arms.

30. The fastener guide of claim 29, wherein said shaft of first guide arm includes a key structure and said first guide arm insert and said shaft of first guide arm inset also include said key structure.

31. The fastener guide of claim 27 wherein said arm is located on the center axis of the fastener guide between the first and second guide arms.

32. The fastener guide of claim 27 wherein said aim is located off the center axis of the fastener guide between the first and second guide arms.

33. The fastener guide of claim 27 wherein said arm is bent one or more times, wherein there will be better visual inspection of the surgical field.

34. A spinal anchoring system for a human spine comprising:
a anchor member having first and second pairs of fasteners each of said fasteners forming an oblique angle with said anchor member, one of said pair of fasteners being a post having an oblique threaded hole therethrough and a second of said pair of fasteners being a screw having a threaded end portion, said threaded portion of said screw being received by said threaded hole and wherein said anchor plate and each of said first and second pairs of fasteners form first and second triangular forms, respectively;
a fastener guide configured to position and to facilitate the placement of each of the first and second pairs of fasteners for said anchor member.

35. The spinal anchoring system of claim 34 wherein said anchor member includes a first plate that includes said first and second opening and said first post and first screw and a second plate slidably coupled to said first plate, said second plate including said third and fourth openings and said second post and screw, wherein said first plate and said first post and said second screw form said first triangular form and said second plate and said second post and said second screw form said second triangular form.
